(19) United States
(12) Patent Application Publication
(10) Pub. No.: US 2018/0021089 A1
(43) Pub. Date: Jan. 25, 2018

(54) Laser Applicator Having Electrodes

(71) Applicant: Vimecon GmbH, Herzogenrath (DE)

(72) Inventors: Kai U. Markus, Eschweiler (DE); Martin Hettkamp, Aachen (DE)

(21) Appl. No.: 15/549,483

(22) PCT Filed: Jan. 25, 2016
(86) PCT No.: PCT/EP2016/051421

§ 371 Date: Aug. 8, 2017

(30) Foreign Application Priority Data
Feb. 9, 2015 (DE) 10 2015 202 214.5

(51) Int. Cl. A61B 18/24

(52) U.S. Cl. CPC A61B 18/24; A61B 2017/00243

(57) ABSTRACT

A laser applicator includes an elongate catheter, which contains at least one peripherally closed lumen, and a light guide, which extends along the catheter and has an outcoupling region in a distal end section of the catheter. The laser applicator has at least one electrode at a distance defined in relation to the outcoupling region such that the position of the outcoupling region in relation to surrounding tissue can be sensed.

The invention relates to a laser applicator having an elongate catheter which contains at least one peripherally closed lumen, and a light guide which extends along the catheter and has an outcoupling region in a distal end section of the catheter.

Such a laser applicator is described in WO 2008/118745 A1 (Vimecon), the disclosure of which is incorporated into the present description by reference. The known laser applicator has an elongate flexible catheter that includes a light guide. The distal end section is formed into a lariat-like loop whose plane extends transversely to the main portion of the catheter. The laser radiation is fed into the light guide at the proximal end. An outcoupling region is located at the distal end of the catheter, where the energy is coupled out laterally from the light guide and leaves the catheter. The distal end is the front end of the catheter directed towards the patient and located opposite the laser energy source. The proximal end is the rear end of the catheter directed towards the laser energy source and located opposite the patient.

The laser applicator serves in particular for the treatment of atrial fibrillation and other cardiac arrhythmia. The applicator may be used to sclerotize heart tissue by converting light energy into thermal energy. The laser radiation leaving the light guide heats the surrounding tissue until a denaturation of proteins occurs and an electrically inactive scar is formed.

DE 10 2006 039 471 B3 describes a laser applicator comprising a catheter with a light guide. In a distal end section of the catheter, the sheath of the light guide has a cutout where light exits laterally from the light guide. Whereas the intact sheath of the light guide causes total reflection, whereby the light energy is transported in the longitudinal direction of the light guide, the cutouts cause refraction at the border of the light guide core, whereby radiation energy is coupled out.

DE 10 2008 058 148 A1 (Vimecon), the disclosure of which is also incorporated into the present description by reference, also describes a laser applicator with an elongate catheter and a light guide extending in the longitudinal direction of the catheter, having an outcoupling region in the distal end section of the catheter.

It is an object of the present invention to provide an improved laser applicator. In particular it is intended to facilitate the navigation of the catheter in the surrounding tissue.

The laser applicator of the present invention is defined by claim 1. According thereto, the laser applicator is provided with electrodes having a defined distance and a defined arrangement with respect to the outcoupling region.

The electrodes are adapted to output an electric signal that is determined by the surroundings of the electrode of the catheter. The electric signal is sensed and evaluated. Based on the electric signal, conclusions can be made as to the electric conductivity of the material surrounding the laser applicator in the region of the electrode and thus to the kind of this material. It may in particular be discriminated whether the electrode is in contact with or surrounded by air, a liquid or biological tissue. Further, the electric signals provide information about the type of the biological tissue. Thus, it is possible to discriminate e.g. between blood, heart muscle and connective tissue.

For this purpose all of the electrodes are electrically connected with an evaluation means that detects and evaluates the electric measuring signals. Here, the measuring values are compared to previous measuring values and/or reference values. The reference values may have been selected for certain materials, such as e.g. a first measuring value for air, a second measuring value for water or a liquid and/or a third measuring value for biological tissue. The evaluation means compares the measured value to one or a plurality of reference values and/or previously recorded measuring values and concludes on the material surrounding the electrode on the basis of the comparison.

The outcoupling region of the laser applicator typically extends for a predefined length in the longitudinal direction of the catheter and for a predefined partial circumference in the circumferential direction of the catheter. This allows laser energy to be emitted laterally out of the laser applicator. If at least one electrode has a contact surface that is exposed to the outside or if the electrode is conductive to the outside only in the region of a partial circumference of the catheter, it is possible to determine the relative rotational position of the outcoupling region with respect to the material electrically contacted by the electrode. In this case the portion of the catheter covered by the electrode and the distance of this partial circumference from the outcoupling region must each be known.

The catheter should not have any electrodes in the outcoupling region so as to avoid the outcoupling region from being covered and to avoid the related compromising of the outcoupling of light. Further, electrodes typically containing metal would be heated by the laser light in the outcoupling region, which should be avoided.

It may be advantageous to arrange two electrodes on sides of the outcoupling region that are opposite to each other in the longitudinal direction of the catheter, so as to be able to detect the position of the outcoupling region in the longitudinal direction of the catheter with respect to material surrounding the catheter. Here, as described above, the outcoupling region may cover only a partial circumference of the catheter. As an alternative, this arrangement of electrodes may also be used in an outcoupling region emitting all around.

In case of an outcoupling region emitting only via a partial circumference, at least one electrode may be provided on the portion of the circumference opposite the outcoupling region or on the portion of the circumference of the laser catheter that is identical with the outcoupling region, which electrode is conductive to the outside only over a partial circumference. Using this electrode, it is then possible to determine the relative rotational position of the outcoupling region with respect to the surrounding material.

The at least one electrode may have an exposed contact surface which, depending on the application, extends over the entire circumference of the catheter or only over a partial circumference of the catheter. A circumferentially extending electrode (annular electrode) is also conceivable, which is covered with an insulator for a part of its circumference so that only the remaining partial circumference not covered by the insulator is conductive to the outside.

Two such electrodes, which are only conductive over a partial circumference, may be arranged at a distance from each other so as to be able to determine the torsion of the catheter between the two electrodes. Here, the relative angle of rotation between the electrodes must be known.

The invention makes it possible for the first time to detect the position of a laser applicator with respect to surrounding tissue without using separate auxiliary means. This is of importance in particular since, in use, the catheter is typically introduced into the patient only via a small opening in the body (“key hole”). The measuring signals of the electrodes are available to a doctor as auxiliary means for establishing sufficient congruence between the outcoupling region and the tissue to be ablated.

An embodiment of the invention will be explained in detail hereunder. In the Figures:

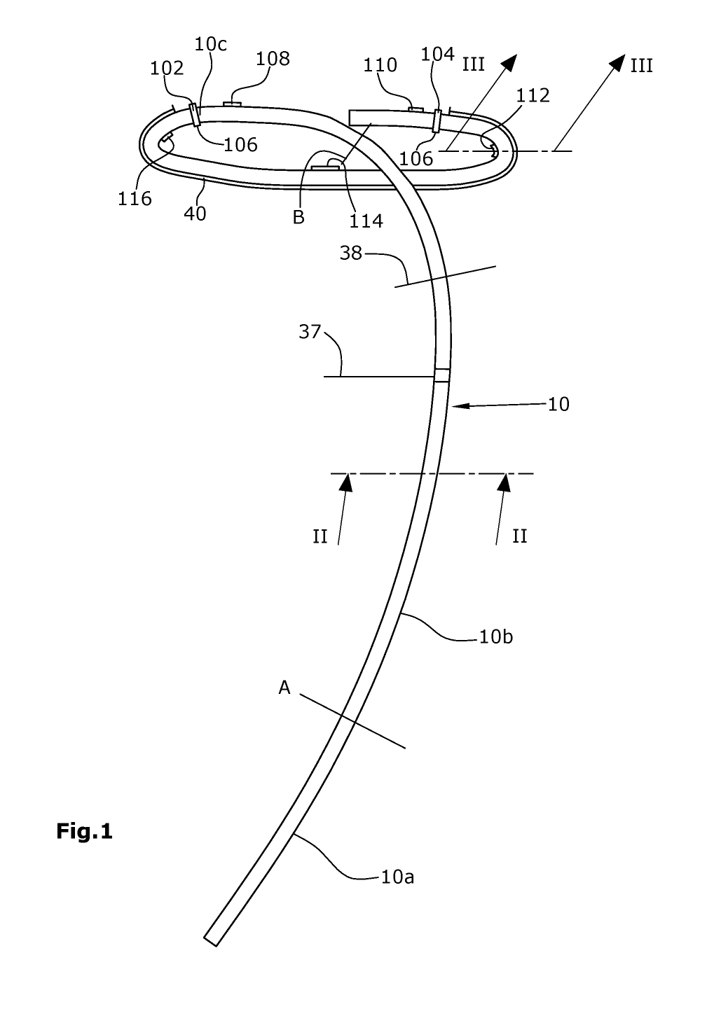

FIG. 1 is a schematic illustration of the general structure of the light guide,

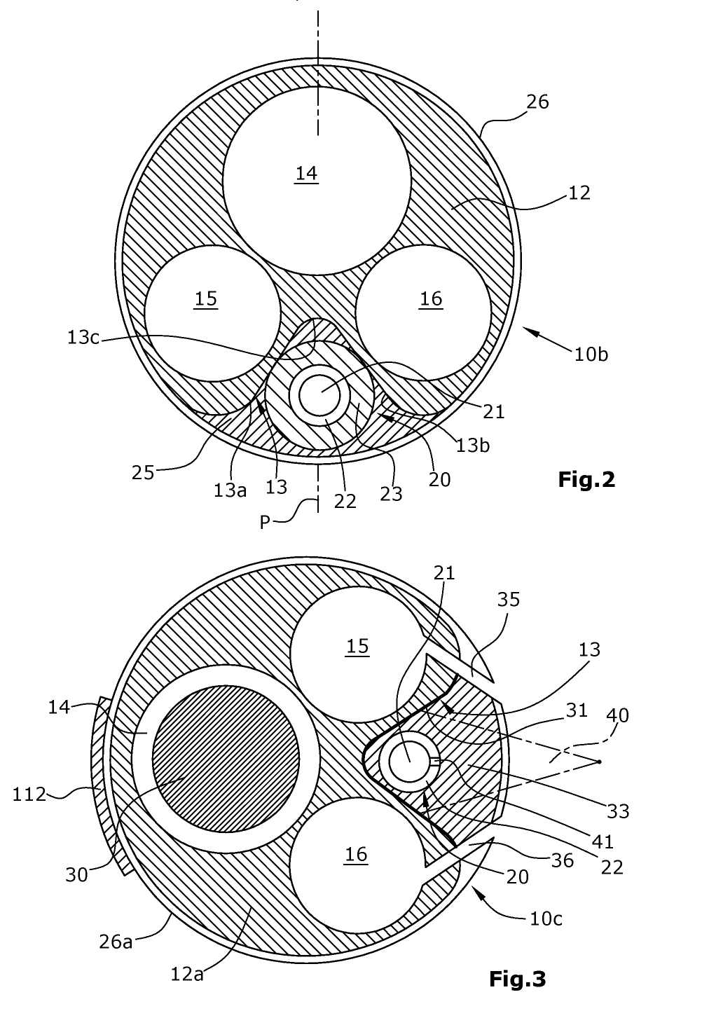

FIG. 2 is a cross section along line II-II in the midsection of the catheter in FIG. 1,

FIG. 3 is a cross section along line III-III in the distal end section of the catheter in FIG. 1,

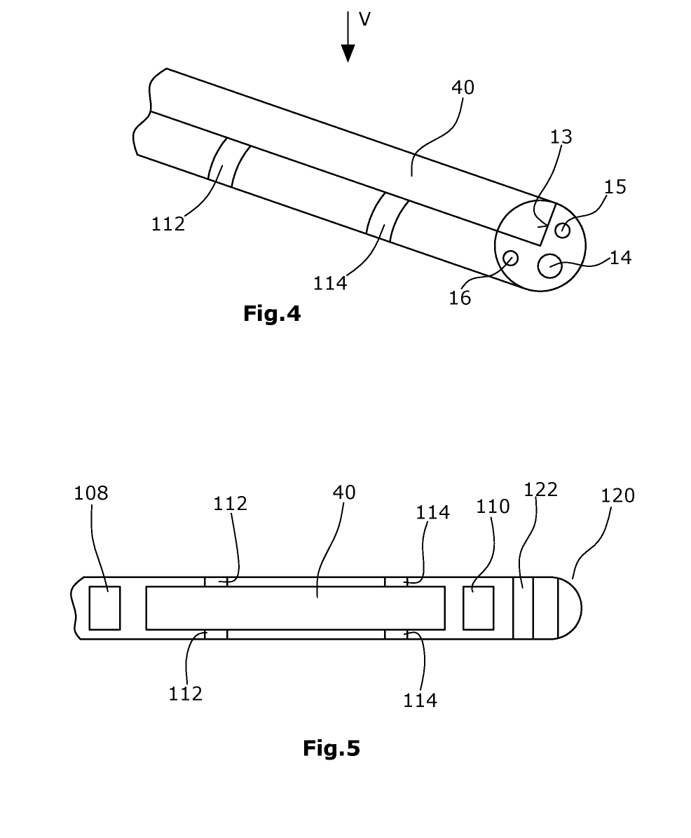

FIG. 4 is a perspective view of another embodiment and

FIG. 5 is a top plan view on the other embodiment in the direction of the arrow V in FIG. 4.

The laser applicator illustrated in the Figures is described for most of its details in DE 10 2008 058 148 A1, the disclosure of which is incorporated into the present description by reference. In the Figures the following elements are identified by reference numerals:

10 catheter

10a section

10b midsection, sections

10c end section

12 catheter body

12a catheter body

13 groove

13a flanks

13b flanks

13c base

14 lumen

15 cooling channels

16 cooling channels

20 light guide

21 core

22 sleeve

23 protective sheath

25 adhesive

26 covering tube

26a covering tube

30 shaping wire

31 reflection layer

33 material

35 outlet bores

36 outlet bores

37 catheter splice site

38 tube splice site

40 outcoupling region

41 openings

The disclosure beyond DE 10 2008 058 148 A1 will be explained in the following:

The length of the outcoupling region in the longitudinal direction of the catheter is identified by the reference numeral 40 in FIG. 1. An annular electrode 102, 104 is arranged proximally of the outcoupling region 40 and distally of the outcoupling region 40, respectively. The annular electrodes 102, 104 are characterized in that they are formed along the circumference of the laser applicator with an electric contact surface 106 so as to be electrically conductive over the entire circumference. Based on the measuring signals of the two annular electrodes 102, 104, it is possible to determine the position of the outcoupling region 40 in the longitudinal direction of the catheter with respect to tissue contacted by the electrodes 102, 104. Further, these annular electrodes are visible in radioscopy and mark the beginning and the end of the outcouplig region 40.

Moreover, a point electrode 108, 110 is arranged proximally and distally of the outcoupling region, respectively. In the circumferential direction of the catheter, the contact surfaces of the two point electrodes 108, 110 are on the same position if the catheter is not twisted. In this case, the relative angle between the two radial lines from the centre of the catheter through the centre of the respective electrode 108, 110 is 0 degrees. As soon as the catheter is twisted by torsion, the relative angle of rotation between the two electrodes 108, 110 changes. If this angle of rotation differs from 0, the catheter experiences torsion. The degree of the torsion of the catheter may be detected by means of the point detectors 108, 110.

The term “point electrode” presently generally denotes a single electrode, wherein the term “point” should not be understood in a mathematical sense. Rather, “point electrode” refers to an electrode that is to pick up the signal at a single point or in a closely confined region of the tissue. The point electrode may be of a circular or disc-shaped design.

In the side of the catheter opposite the outcoupling region 40 further electrodes 112, 114, 116 are arranged which each cover only a partial circumference of the catheter in an electrically conductive manner. Using these electrodes 112, 114, 116 it is possible to determine the relative angle of rotation of the outcoupling region 40 with respect to the tissue contacted by the electrodes 112, 114, 116. In particular, these electrodes 112, 114, 116 serve to determine the contact of the outcoupling region 40 with tissue, such as heart muscle tissue. This is because the electrodes on the rear of the outcoupling region should not be in contact with tissue, as long as the outcoupling region is in contact with tissue.

In the embodiment of FIGS. 4 and 5 two electrodes 112, 114 are arranged at a distance from each other on the side of the catheter opposite the outcoupling region 40. These electrodes are each designed as annular electrodes that cover the circumference mot covered by the outcoupling region 40. The circumference portion of the outcoupling region 40 is thus cutout in each of the two electrodes 112, 114. If one of the electrodes 112, 114 sends an electric signal due to contact with tissue, this may be seen as an indication that, in the region of the respective electrode, the outcoupling region 40 has no contact with tissue.

Further, an electrode 108 is arranged proximally of the outcoupling region 40 and another electrode 110 is arranged distally of the outcoupling region 40 on e same side of the catheter as the outcoupling region 40. The two electrodes 108, 110 each cover the same circumferential section of the catheter as the outcoupling region 40. If both electrodes 108, 110 send an electric signal caused by contact with tissue, this may be seen as an indication that the outcoupling region 40 situated therebetween is also in contact with tissue.

In the embodiment shown in FIG. 5, a distal end electrode 120 is formed as a full electrode at the distal end of the catheter. This means that the electrode 120 entirely covers the distal end face of the catheter. The electrode 120 of this embodiment is of a spherical-cap shape so as to form a blunt catheter end.

Proximally of the full electrode 120, an annular electrode 122 is arranged at a small distance of a few millimeters (less than 1 cm). The annular electrode 120 is a full electrode covering the full circumference of the catheter.

1. A laser applicator comprising an elongate catheter which contains at least one peripherally closed lumen, and a light guide, which extends along the catheter and has an outcoupling region in a distal end section of the catheter, wherein
said laser applicator has at least one electrode at a distance defined in relation to the outcoupling region such that the position of the outcoupling region in relation to surrounding tissue can be sensed.

2. The laser applicator of claim 1, wherein the electrode has a con-tact surface exposed to the outside.

3. The laser applicator of claim 2, wherein the electrode has a contact surface exposed to the outside only in the region of a partial circumference, so as to allow the determination of the relative position of rotation of the outcoupling region with respect to contacted tissue, the outcoupling region also extending only over a partial circumference in the circumferential direction of the catheter.

4. The laser applicator of claim 1, wherein the electrode is arranged in the region of the distal end section.

5. The laser applicator of claim 1, wherein at least one electrode is arranged distally of the outcoupling region.

6. The laser applicator of claim 1, wherein two electrodes are arranged on sides of the outcoupling region opposing each other in the longitudinal direction of the catheter, so as to be able to sense the position of the outcoupling region in the longitudinal direction of the catheter with respect to surrounding tissue.

7. The laser applicator of claim 1, wherein on the side of the laser catheter opposite the outcoupling portion, at least one electrode is arranged which is conductive to the outside only over a partial circumference of the catheter.

8. The laser applicator of claim 1, wherein at least one electrode is formed to extend circumferentially in the circumferential direction of the catheter and, for a part of the circumference, is covered with an insulator towards the outside.

9. The laser applicator of claim 8, wherein at least two electrodes, which are formed to extend circumferentially in the circumferential direction and covered with an insulator towards the outside for a part of the circumference, are arranged at a distance from each other so as to allow the determination of the torsion of the catheter between the two electrodes.

10. The laser applicator of claim 1, wherein an evaluation means is provided for receiving and evaluating the electric signal, the evaluation means being electrically connected with each electrode.

11. The laser applicator of claim 1, wherein proximally and/or distally of the outcoupling region an electrode is respective arranged on the same side of the catheter as the outcoupling region.

12. The laser applicator of claim 11, wherein the electrodes arranged proximally and distally of the outcoupling region cover the same circumference as the outcoupling region.

13. The laser applicator of claim 1, wherein a fully circumferentially extending annular electrode and/or an electrode covering the distal end face of the catheter are arranged at the distal end of the catheter.

14. A method for determining the relative position of the outcoupling region of a laser applicator according to claim 1, with respect to material electrically contacted by the electrodes, comprising the following steps:
measuring the electric potential or an electric current of the electrode,
comparing the measured value to a reference value or to a previous measuring value, and
determining, based on the comparison, whether the electrode electrically contacts electrically conductive material.

15. The method of claim 14, wherein the reference value is a value that corresponds to a case in which the electrode is in contact with an electric insulator, e.g. air, or to a case in which the electrode is in contact with electrically conductive material, e.g. biological tissue or water.
